(19) United States
(12) Patent Application Publication
(10) Pub. No.: US 2021/0401094 A1
(43) Pub. Date: Dec. 30, 2021

(54) VENTRICULAR ASSIST DEVICE CONTROLLER HOLDING GARMENT ASSEMBLY

(71) Applicant: Deborah Clemens, Fairmont, WV (US)

(72) Inventor: Deborah Clemens, Fairmont, WV (US)

(21) Appl. No.: 16/917,097

(22) Filed: Jun. 30, 2020

(51) Int. Cl. A41D 13/12; A41D 27/20

(52) U.S. Cl. CPC A41D 13/1245; A41D 27/205; A61M 1/10; A41D 2300/324; A41D 27/201

(57) ABSTRACT

A ventricular assist device controller holding garment assembly includes a shirt that has a front side, a back side, a first lateral side, a second lateral side, a right sleeve, and a left sleeve. The shirt has a neck opening and a torso opening and bounds an interior space. A front pocket is positioned on an outer surface of the front side of the shirt. The front pocket is positioned nearer to the torso opening than the neck opening and has an upper edge comprising an access opening of the front pocket. The front pocket has an exterior wall. A shield comprising a heat resistant material is positioned between the exterior wall and the interior space. A controller receiving space is defined between the exterior wall and the shield and holds a controller for a ventricular assist device.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to medical controller holding garment devices and more particularly pertains to a new medical controller holding garment devices for holding articles required by a wearer of the garment for running necessary medical treatment devices.

### (2) Description of Related Art Including Information Disclosed Under 37 CFR 1.97 and 1.98

The prior art relates to medical garment assemblies that are used for holding automated medical treatment devices.

### BRIEF SUMMARY OF THE INVENTION

An embodiment of the disclosure meets the needs presented above by generally comprising a shirt that has a front side, a back side, a first lateral side, a second lateral side, a right sleeve, and a left sleeve. The shirt has a neck opening and a torso opening and is comprised of a fabric material. The shirt bounds an interior space. A front pocket is positioned on an outer surface of the front side of the shirt. The front pocket is positioned nearer to the torso opening than the neck opening and has an upper edge comprising an access opening of the front pocket. The front pocket has an exterior wall. A shield comprising a heat resistant material is positioned between the exterior wall and the interior space. A controller receiving space is defined between the exterior wall and the shield and holds a controller for a ventricular assist device.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a ventricular assist device controller holding garment assembly according to an embodiment of the disclosure.

FIG. 2 is a front view of an embodiment of the disclosure.

FIG. 3 is a front isometric view of an embodiment of the disclosure.

FIG. 4 is a cross-sectional view of an embodiment of the disclosure taken along line 4-4 of FIG. 2.

FIG. 5 is a front view of an embodiment of the disclosure.

FIG. 6 is a side view of an embodiment of the disclosure.

### DETAILED DESCRIPTION OF THE INVENTION

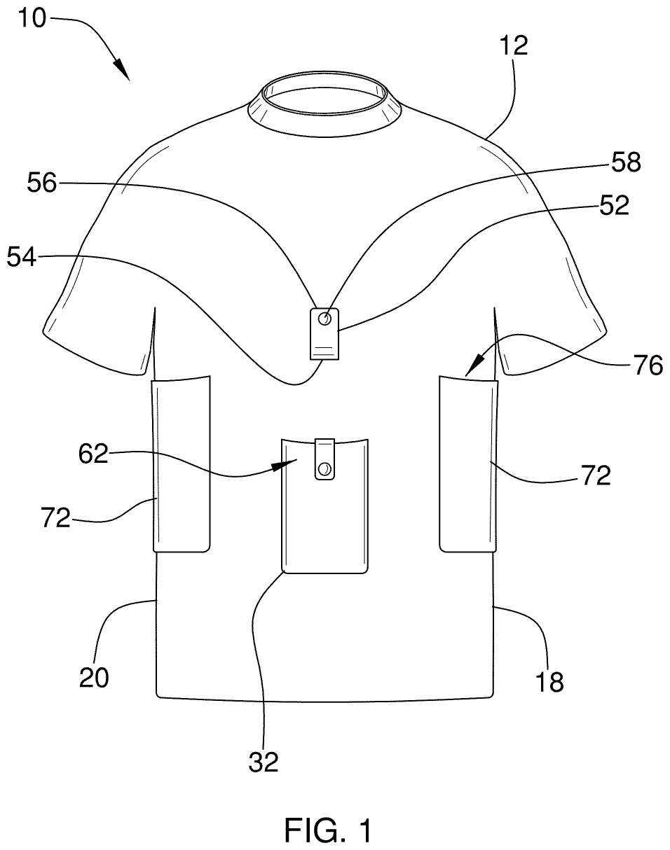

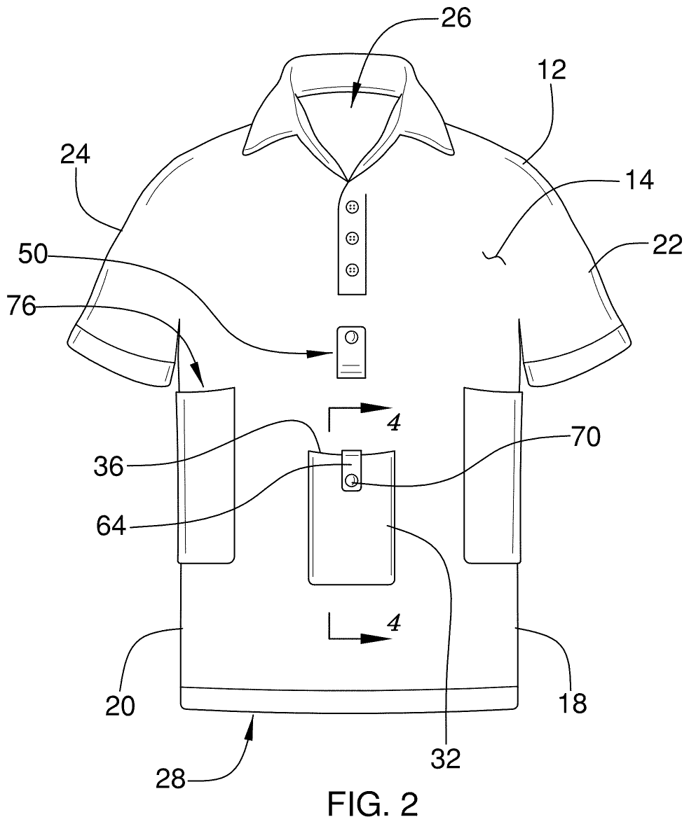

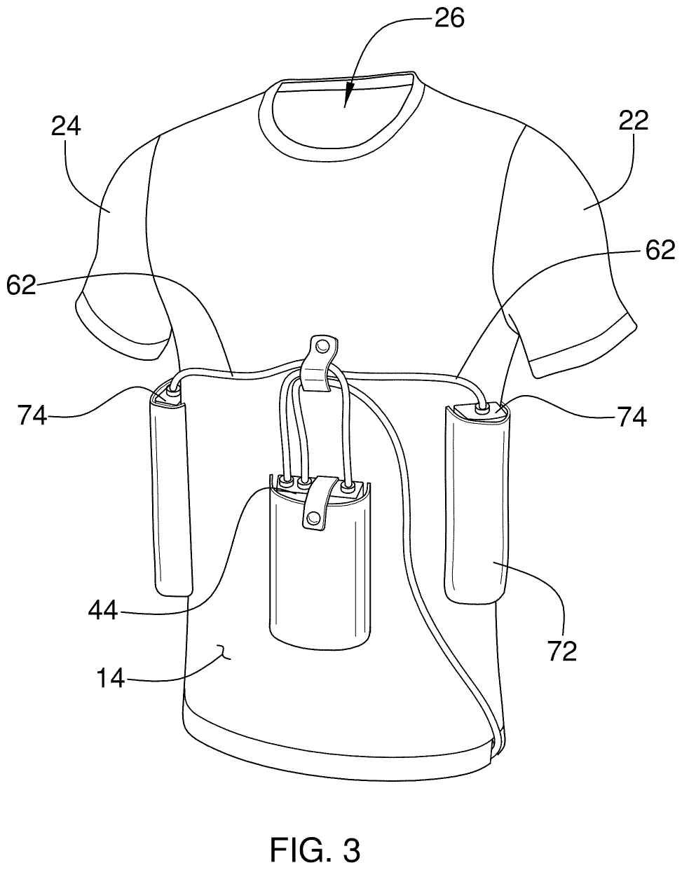

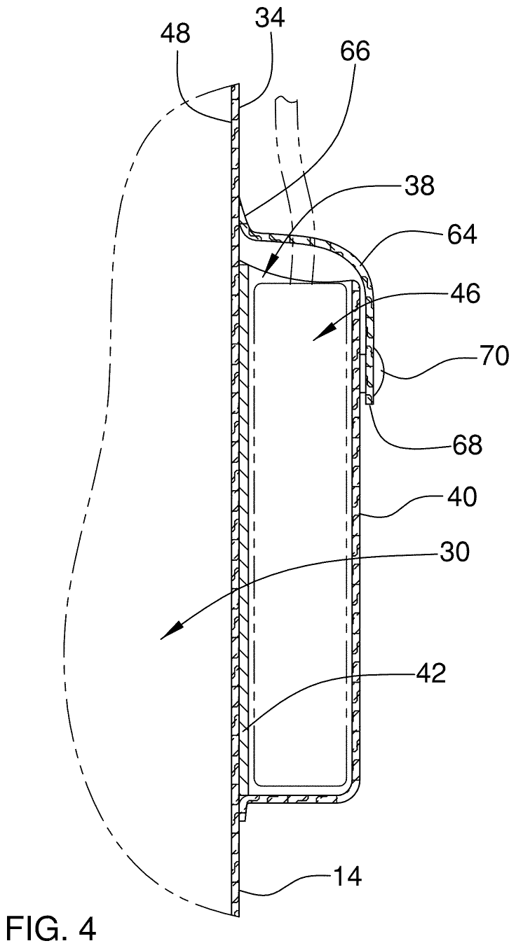

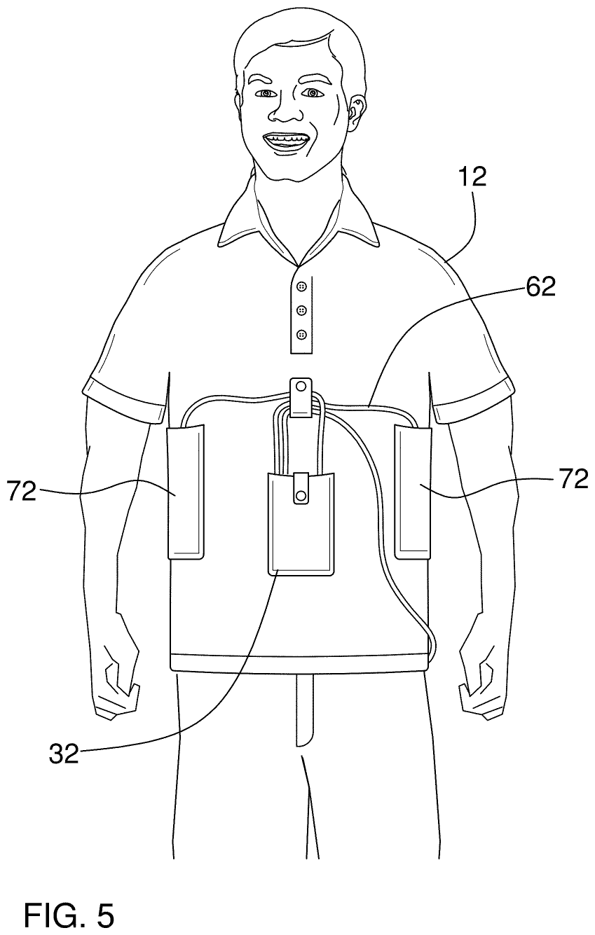

With reference now to the drawings, and in particular to FIGS. 1 through 6 thereof, a new medical controller holding garment devices embodying the principles and concepts of an embodiment of the disclosure and generally designated by the reference numeral 10 will be described.

As best illustrated in FIGS. 1 through 6, the ventricular assist device controller holding garment assembly 10 generally comprises a shirt 12 that has a front side 14, a back side 16, a first lateral side 18, a second lateral side 20, a right sleeve 22, and a left sleeve 24. The shirt 12 has a neck opening 26 and a torso opening 28. The shirt 12 is generally conventional and is comprised of a fabric material. The fabric material may include cotton, cotton blends, synthetic materials, silks and the like. The shirt 12 may include a T-shirt, polo type shirt and the like and will be comprised of materials that are preferably breathable and soft. The shirt 12 may be lined with, or comprised of, a fleece material, for example, to impart softness though breathability will be lowered utilizing such. The shirt 12 bounds an interior space 30.

A front pocket 32 is positioned on an outer surface 34 of the front side 14 of the shirt 12. The front pocket 32 is positioned nearer to the torso opening 28 than the neck opening 26 and is generally laterally centered on the front side 14. The front pocket 32 has an upper edge 36 comprising an access opening 38 of the front pocket 32. The front pocket 32 is comprised typically of a fabric material any may be comprised of same material as the shirt 12. The front pocket 32 has an exterior wall 40.

A shield 42 comprising a heat resistant material is positioned between the exterior wall 40 and the interior space 30. The purpose of the shield 40 is to protect the wearer of the garment assembly 10 from heat produced by a ventricular assist device controller 44. The shield 42 may be comprised of any heat resistant material, though flexible materials are preferred to aid in the comfort of the wearer. Therefore, materials used for conventional fabric potholders, for example, may be utilized. However, synthetic materials such as silicone panels may also be utilized. A controller receiving space 46 is defined between the exterior wall 40 and the shield 42 and is configured to receive the controller 44 for a ventricular assist device. In some embodiments, the shield 42 may be attached to the front side 14 of the shirt 12 within the front pocket 32. Alternatively, the shield 42 may be removably positioned within the front pocket 32 such that it may be cleaned separately or removed when the shirt 12 is cleaned. If the shield 42 is comprised of a soft, flexible material, the shield 42 may be attached 48 to an inner surface of the front side.

A cord retaining member 50 is attached to the front side 14 and is positioned between the front pocket 32 and the neck opening 26. The cord retaining member 50 includes a flap 52 that has an attached end 54 and a loose end 56. The loose end 56 is removably secured to the front side 14 with a coupler 58. The cord retaining member 50 receives cords 60 from the ventricular assist device 44 wherein the cords 60 are positionable between the flap 52 and the shirt 12. The coupler 58 may comprise hook and look couplers, a button, and the like, however, it may be preferred that the coupler 58 comprises a snap due to its greater mechanical strength. The flap 52 may be comprised of a fabric or elastomeric material.

A front pocket closure 62 may be provided to releasably close the access opening 38. The front pocket closure 62 includes a tab 64 that has a first end 66 attached to the front side 12 and a second end 68 that is removably attached to the exterior wall 40 with a fastener 70 such that it extends over the access opening 38 to prevent the controller 44 from falling out of the front pocket 32. The fastener 70 may include the same types of items used for the coupler 58 but may again be preferred if the fastener 70 comprises a snap.

At least one battery pocket 72 is mounted on the shirt 12 and is positioned lateral to the front pocket 32. Ventricular assist devices, to be mobile, include batteries 74 for supplying power to the control assembly 44 and to a pump controlled by the control assembly 44. The at least one battery pocket 72 is configured to be receive a battery 74 electrically coupled to the controller 44. The at least one battery pocket 72 will typically include a first battery pocket 72 positioned on the first lateral side 18 and a generally identical second battery pocket 72 positioned on the second lateral side 20. The battery pockets 72 each have an open top end 76 for receiving the batteries 74. Though not shown, the battery pocket(s) 72 may also include closing members having the same structure as the front pocket closure 62.

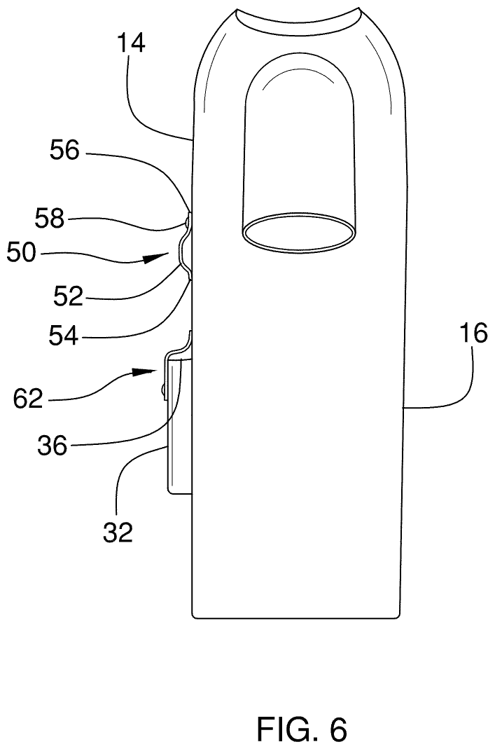

FIG. 6 depicts an embodiment of the invention wherein the shirt 12 does not include any battery pockets 72. This embodiment it utilized for sleeping, in particular, and may include multiple ones of cord retaining members 50 to prevent the cords 62 from tangling when the wearer moves during their sleep. This embodiment allows usage of elongated power cords which are used while the batteries 74 are being recharged.

In use, the shirt 12 is worn by a person who must support the control assembly 44, and possibly batteries 74, in a manner that prevents the control assembly 44 from falling outwardly away from the shirt 12 while also protecting the user from the heat of the control assembly 44. Aside from enhancing comfort for the wearer, the shield 42 reduces the risk of infection at the control cord entrance point into the wearer's body by preventing heat from being applied to or near the entrance point.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A garment assembly configured for securely holding a ventricular assist device controller, the garment comprising:
a shirt having a front side, a back side, a first lateral side, a second lateral side, a right sleeve, and a left sleeve, the shirt having a neck opening and a torso opening, the shirt being comprised of a fabric material, the shirt bounding an interior space;
a front pocket being positioned on an outer surface of the front side of the shirt, the front pocket being positioned nearer to the torso opening than the neck opening, the front pocket having an upper edge comprising an access opening of the front pocket, the front pocket having an exterior wall; and
a shield comprising a heat resistant material being positioned between the exterior wall and the interior space, a controller receiving space being defined between the exterior wall and the shield and being configured to receive a controller for a ventricular assist device.

2. The garment assembly configured for securely holding a ventricular assist device controller according to claim 1, wherein the shield is attached to the front side of the shirt.

3. The garment assembly configured for securely holding a ventricular assist device controller according to claim 1, further including a cord retaining member being attached to the front side and being positioned between the front pocket and the neck opening.

4. The garment assembly configured for securely holding a ventricular assist device controller according to claim 3, wherein the cord retaining member includes a flap having an attached end and a loose end, the loose end being removably secured to the front side with a coupler, wherein cords from the ventricular assist device are positionable between the flap and the shirt.

5. The garment assembly configured for securely holding a ventricular assist device controller according to claim 4, wherein the coupler comprises a snap.

6. The garment assembly configured for securely holding a ventricular assist device controller according to claim 1, further including a front pocket closure releasably closing the access opening.

7. The garment assembly configured for securely holding a ventricular assist device controller according to claim 6, wherein the front pocket closure includes a tab has a first end being attached to the front side and a second end being removably attached to the exterior wall with a fastener.

8. The garment assembly configured for securely holding a ventricular assist device controller according to claim 7, wherein the fastener comprising a snap.

9. The garment assembly configured for securely holding a ventricular assist device controller according to claim 3, wherein the front pocket closure includes a tab has a first end being attached to the front side and a second end being removably attached to the exterior wall with a fastener.

10. The garment assembly configured for securely holding a ventricular assist device controller according to claim 9, wherein the fastener comprising a snap.

11. The garment assembly configured for securely holding a ventricular assist device controller according to claim 1, further including at least one battery pocket being mounted on the shirt and being positioned lateral to the front pocket, the at least one battery pocket being configured to be receive a battery electrically coupled to the controller.

12. The garment assembly configured for securely holding a ventricular assist device controller according to claim 11, wherein the at least one battery pocket includes a first battery pocket positioned on the first lateral side and a second battery pocket positioned on the second lateral side.

13. The garment assembly configured for securely holding a ventricular assist device controller according to claim 6, further including at least one battery pocket being mounted on the shirt and being positioned lateral to the front pocket, the at least one battery pocket being configured to be receive a battery electrically coupled to the controller.

14. The garment assembly configured for securely holding a ventricular assist device controller according to claim 13, wherein the at least one battery pocket includes a first battery pocket positioned on the first lateral side and a second battery pocket positioned on the second lateral side.

15. A garment assembly configured for securely holding a ventricular assist device controller, the garment comprising:
a shirt having a front side, a back side, a first lateral side, a second lateral side, a right sleeve, and a left sleeve, the shirt having a neck opening and a torso opening, the shirt being comprised of a fabric material, the shirt bounding an interior space;
a front pocket being positioned on an outer surface of the front side of the shirt, the front pocket being positioned nearer to the torso opening than the neck opening, the front pocket having an upper edge comprising an access opening of the front pocket, the front pocket having an exterior wall;
a shield comprising a heat resistant material being positioned between the exterior wall and the interior space, a controller receiving space being defined between the exterior wall and the shield and being configured to receive a controller for a ventricular assist device, the shield being attached to the front side of the shirt;
a cord retaining member being attached to the front side and being positioned between the front pocket and the neck opening, the cord retaining member including a flap having an attached end and a loose end, the loose end being removably secured to the front side with a coupler, wherein cords from the ventricular assist device are positionable between the flap and the shirt, the coupler comprising a snap;
a front pocket closure releasably closing the access opening, the front pocket closure including a tab having a first end being attached to the front side and a second end being removably attached to the exterior wall with a fastener, the fastener comprising a snap; and
at least one battery pocket being mounted on the shirt and being positioned lateral to the front pocket, the at least one battery pocket being configured to be receive a battery electrically coupled to the controller, the at least one battery pocket including a first battery pocket positioned on the first lateral side and a second battery pocket positioned on the second lateral side.
